(19) United States
(12) Patent Application Publication
(10) Pub. No.: US 2009/0155332 A1
(43) Pub. Date: Jun. 18, 2009

(54) REPLACEMENT BONE TISSUE

(76) Inventors: Eugene Sherry, New South Wales (AU); Sureshan Sivananthan, London (GB); Patrick Warnke, Augustfehn (DE)

Correspondence Address: KOHN & ASSOCIATES, PLLC, 30500 NORTHWESTERN HWY, SUITE 410, FARMINGTON HILLS, MI, 48334, US

(21) Appl. No.: 11/817,158

(22) PCT Filed: Feb. 24, 2006
(86) PCT No.: PCT/AU06/00228

§ 371 Date: Oct. 27, 2008

(30) Foreign Application Priority Data
Feb. 25, 2005 (AU) 2005900884

(51) Int. Cl. A61F 2/28; A61K 35/12

(52) U.S. Cl. 424/423; 424/93.7

(57) ABSTRACT

Bone replacement tissue suitable for bone grafting procedures, said bone replacement tissue being grown in a host until suitable for translocation into a desired position in a patient, and methods for manufacturing said bone replacement tissue.

### FIELD OF THE INVENTION

The present invention relates to replacement tissue, and in particular, to replacement bone material suitable for use in bone grafting procedures, and to methods for manufacturing said replacement bone material.

### BACKGROUND OF THE INVENTION

Bone tissue is composed of a matrix that primarily consists of collagen protein, but is strengthened by deposits of calcium, hydroxyl and phosphate salts, referred to as hydroxyapatite. Inside and surrounding this matrix lie the cells of bone tissue, which include osteoblasts, osteocytes, osteoclasts and bone-lining cells. All four of these cell types are required for building and maintaining a healthy bone matrix, as well as remodelling of the bone under certain conditions.

Injury, disease and developmental defects can all result in bone defects that require bone grafting procedures, where new bone or a replacement material is placed in apertures around a fractured bone, or holes/defects in bone. Bone grafting assists the bone to heal, or merely provides mechanical structure to the defective bone, through the provision of artificial material that is not incorporated into a patient's own bone. Bone grafts can be osteogenic, osteoconductive or osteoinductive.

Autografting may be used where it is appropriate to take the patient's own bone tissue from another site in the body, usually the iliac crest, although the distal femur and proximal tibia may also be used. Autografting has advantages in terms of its provision of osteoconductivity (ie. the graft supports the attachment of new osteoblasts and osteoprogenitor cells). Furthermore, it provides osteoinductivity, or the ability to induce nondifferentiated cells into osteoblasts.

In the context of autografting for injuries such as bone fractures, the grafting procedure can be quite complex, and may fail to heal properly. Grafting for bone fractures is generally only considered when a reasonable sized portion of bone has been lost via fracture. In this context, bone grafting may be performed using the patient's own bone, usually taken from the hip, or using bone from a donor. The donor/replacement bone is usually held in place by physical means (eg. screws and pins), while the healing process occurs.

The drawbacks of autografting procedures include surgical complications (eg. acute and chronic pain, inflammation, infection), and limitations in relation to the amount of bone that can be harvested for grafting. Furthermore, complications occurring after bone grafting include fracture at the donor site after cortical graft removal, intraoperative bleeding and postoperative pain after iliac crest biopsy and stress fractures, hernias through an iliac donor site and gait problems.

An alternative procedure, allografting, where bone graft material is taken from a donor or cadaver, offer some advantages over autografting in terms of the lack of surgical complications in obtaining the bone graft material. However, there is a risk of disease transmission from the donor to the recipient of the bone graft material, which is not overcome by pre-implantation treatment of the bone with techniques such as gamma irradiation. Furthermore, the allograft may not knit well with the patient's own bone, leading to weakness at the point of union of the graft. Also, where bone is harvested from a donor, there exist the same risks as harvesting replacement bone from the patient, as discussed above.

A variety of alternative graft materials exist, including ceramic materials, polymeric materials and chemically inert substances. These bone substitutes are often innoculated with bone marrow and/or growth factors to provide osteoconductive and osteoinductive properties provided by use of autografted bone.

In the case of certain bone substitute materials, there is the disadvantage that they do not become permanently incorporated into a patient's own bone and are thus subject to breakage, loosening, erosion and osteolysis.

Furthermore, while in vivo bone reconstruction using a polymeric matrix has been found to have the capacity for bone regeneration (Borden et al., J Bone Joint Surg Br. 2004 November; 86(8):1200-8; Mankani et al., Biotechnol Bioeng. 2001 Jan. 5; 72(1):96-107), the site of regeneration will naturally be in a weakened state until full bone mineralisation and osteoblast replacement is attained.

Accordingly, there remains a need for improved methods in bone replacement technology.

### BRIEF DESCRIPTION OF THE DRAWINGS

FIG. 1A: schematic diagram showing minimally invasive surgical instrument (MIS) making an incision into a patient's thigh.

FIG. 1B: schematic diagram showing loading of the MIS instrument with the scaffold.

FIG. 1C: schematic representation showing release of the scaffold implant and replacement bone tissue from the MIS instrument.

FIG. 2A: schematic representation showing scaffold and bone replacement material being mobilised using MIS instruments.

FIG. 2B: schematic representation showing movement of scaffold and bone replacement tissue into final location.

FIGS. 3A TO 3C: schematic representations of loading and unloading of cone-shaped scaffold from MIS instrument and

FIGS. 4A TO 4C: schematic representations showing insertion of cone-shaped scaffold into a defect in the upper femur in the clinical situation of bone stock loss after failed joint replacement surgery.

FIG. 5: An example of a double-layered scaffold for a femoral insert suitable for use for in vivo bone growth.

FIG. 6: An example of a double-layered scaffold for a vertebral disc suitable for in vivo bone growth.

FIG. 7: Schematic diagram showing a cone- and cup-shaped scaffolds suitable for joints.

### DESCRIPTION OF THE INVENTION

Throughout this specification, unless the context requires otherwise, the words “comprise,” “comprises” and “comprising” will be understood to imply the inclusion of a stated step or element or group of steps or elements but not the exclusion of any other step or element or group of steps or elements.

The reference to any prior art in this specification is not, and should not be taken as, an acknowledgement or any form of suggestion that the prior art forms part of the common general knowledge in Australia.

It has surprisingly been found by the present inventors that replacement bone material that is highly suitable for bone grafting can be grown in vivo when a bone scaffold containing osteoblast progenitor cells is placed in certain tissues in a host close to the site where replacement bone material is required. Thus placed, the bone scaffold undergoes osteogenesis and angiogenesis of the newly formed bone tissue, and can be translocated to the site where the replacement bone material is required without substantial disruption to the replacement bone's blood supply. This is described by the inventors as in-vivo engineering of bone in a living bioreactor or in another preferred form, bone endocultivation.

Accordingly, in a first broad form, the present invention relates to a method for growing replacement bone tissue for a patient, said method comprising:
a) providing a scaffold or gel for the replacement bone tissue to grow into.
b) inoculating the scaffold with osteoblast precursor cells;
c) implanting the scaffold in a location close to a site where replacement bone is required, wherein the location comprises a subcutaneous compartment, a myofascial or fat tissue or a subperiosteal space.
d) allowing osteogenesis and angiogenesis of the replacement bone tissue;
e) relocating the replacement bone tissue and its substantially intact blood supply to the site where replacement bone is required in the patient.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

Preferably, the scaffold is filled with bone minerals (ie. hydroxyapatite), such as in the form of bone mineral crystals or blocks, which serve as hydroxyapatite carriers for cells and growth factors that are added to the cage. Suitable bone hydroxyapatite materials are known to those skilled in the art and include, for example, allograft-based bone graft substitutes in which allograft bone is used alone or along with other materials (eg. Allogro, Othroblast, Opteform, Grafton, VG1 ALIF, VG2 PLIF, geneX). Alternatively, ceramic materials may be used, which may be bioactive and/or resorbable (eg. calcium phosphate, bioglass plus Osteograf, Norian SRS, ProOsteon, Osteoset, Ossatura, Cerasorb, Chronos, BonePlast, Novabone, Novamin), or polymeric materials that may or may not be biodegradable, plus (eg. polymer of degradable or non-degradable synthetic collagen fiber/felted mass. (eg. Cortoss, Immix, Infuse, Healos (collagen with HA coating).

In another preferred form, the scaffold is a gel or liquid based.

The osteoblast precursor cells that are used to inoculate the scaffold may be provided by a mixture of bone marrow cells, as bone marrow is known to contain mesenchymal stem cells and haematopoietic stem cells, both of which have an ability to differentiate into osteoblasts. Alternatively, the bone marrow mixture may be purified in order to provide a concentrated mix of either or both of these stem cells types.

The osteoblast precursor cells may be isolated from the patient and thus autogenic relative to the patient's own tissue, or may be isolated from another organism, and thus allogenous with respect to the patients own tissues.

In one embodiment, stem cells may be totipotent stem cells isolated from fertilised eggs from the host species, adult stem cells or pluripotent stem cells which are embryonic stem cells isolated from the host species.

In a further form, the stem cells may be adult stem cells harvested from the patient or a stem-cell donor.

In a particularly preferred form, the scaffold is also inoculated with at least one growth factor on an outer and/or inner surface of the scaffold, wherein the inner surface of the scaffold represents the surface housing the bone minerals. Preferably, the at least one growth factor is an osteoblast growth factor, and even more preferably, a bone morphogenetic protein (BMP). The BMPs are a group of related proteins originally identified by their presence in bone-inductive extracts of demineralized bone. Molecular cloning has revealed at least six related members of this family, which have been designated BMP-2 through BMP-7. These molecules are part of the TGF-β superfamily.

The BMPs can be divided into subgroups with BMP-2 and BMP-4 being 92% identical, and BMP-5, BMP-6, and BMP-7 being an average of about 90% identical. Single BMP molecules, such as BMP-2, are capable of inducing the formation of new cartilage and bone (Li et al., J Spinal Disord Tech. 2004 October; 17(5):423-8). Whether each of the BMPs possesses the same inductive activities in an animal is the subject of ongoing research. Studies of transgenic and knockout mice and from animals and humans with naturally occurring mutations in BMPs and related genes have shown that BMP signaling plays critical roles in heart, neural and cartilage development (Chen et al., Growth Factors. 2004 December; 22(4):233-41).

In one preferred form, the BMP is BMP-7. In an even more preferable form, the BMP is BMP-2

In one preferred form, the scaffold is a cone shape or a cup shape. In this embodiment, these scaffolds may be used to replace lost or damaged bone resulting from failed hip or knee joint reconstruction or replacement.

In another preferred form, anatomical modelling studies are performed prior to shaping the scaffold, in order to produce a scaffold having a shape that is optimised to fit into the region where the replacement bone material is required. A variety of techniques exist in the art and are well known by the skilled person, such as computed tomography and magnetic resonance imaging, which are preferably assisted by the use of computer-aided design.

In a preferred form, the scaffold is a suitable biocompatible and/or bioabsorbable material. A variety of suitable biocompatible and/or bioabsorbable materials are known in the art, and include, but are not limited to, titanium, stainless steel, zirconium oxide, ceramic tricalcium phosphate and polymers. In a particularly preferred form, the cage is formed from titanium or tantalum or an alloy.

Even more preferably, the scaffold has an inner, mesh-like surface that provides an inner compartment, and an outer, substantially continuous surface separated from the inner, mesh-like surface, the outer surface having at least one aperture through which osteoblastic precursor cells, growth factors, bone crystals or bone paste may be injected by passing through the at least one aperture to the inside of the outer layer of the scaffold.

Even more preferably, the bilayered scaffold is made from titanium.

The present invention has an advantage in that a nutrient supply is provided by placement of the bone scaffold into an area that is close to the bone that is going to be replaced, which permits osteogenesis of the bone replacement tissue to occur, as well as angiogenesis to the bone replacement tissue.

The anatomical regions into which the scaffold may be implanted include subcutaneously, into fat tissue or into a muscle.

As briefly mentioned, the vascular supply of the subcutaneous region, the fat or the muscle containing the scaffold assists in angiogenesis of the newly grown bone. Accordingly, the newly growing bone is provided with a blood supply, and most of the blood vessels that grow into the bone graft tissue can remain attached to the bone graft tissue when it is translocated into its new location, due to its proximity to the bone of the patient that is to be replaced in a bone grafting procedure. This translocation can occur once sufficient bone formation has occurred within the scaffold.

The term “sufficient bone formation” as used herein refers to the formation within the scaffold of adequate amounts of bone, as determined by extent of mineralisation and bone formation (ie. formation of osteoblasts and trabeculae) within the titanium scaffold, as determined by any suitable methods in the art, such skeletal scintigraphy, as x-ray analysis and computerised tomography.

In yet another first broad form, the present invention relates to a method for growing bone for a bone graft in a patient, wherein the method involves the steps of:
a) providing a scaffold for the replacement bone tissue;
b) inoculating the scaffold with osteoblast precursor cells;
c) implanting the scaffold to a site where replacement bone tissue is required in the patient.

The method according to this embodiment is particularly useful where clinical situations involving loss of bone stock has occurred following (failed) joint replacement surgery. For example, in the situation of a failed hip or knee replacement/reconstruction, cone- or cup-shaped scaffolds can be placed in situ, ie used to fill the bone stock loss (defect) of the upper or lower femur or the ball and socket joint (acetabulum) of the femoral head (FIG. 7).

In a further broad form, the present invention relates to a bone growth paste, which supports formation of new bone. The bone growth paste contains osteoblast precursor cells, bone crystals and at least one growth factor.

Preferably, the at least one growth factor is an osteoblast growth factor.

Preferably, the bone crystals, which may be hydroxyapatite crystals, are shaped prior to inclusion in the bone paste, and even more preferably, the bone crystals are hexagonally shaped. In one preferred form the bone crystals are shaped using selective laser melting.

In another preferred form they are shaped using selective laser sintering technology. In yet another preferred form the bone crystals are shaped using boundary element methods.

In yet a further form, the invention relates to a kit for growing replacement bone for a patient, the kit comprising:
a) a growth guiding biomimetic scaffold which may or may not be computer designed, suitable for supporting bone growth subcutaneously, subfascially, subperiosteally or within a fat or muscle pouch of a patient; and
b) a device used to harvest or collect or otherwise generate a volume or aliquot of cells which may be stem cells, osteoblast precursor cells, chondrocyte cells or cord blood cells.
c) A suitably preserved volume of cells for inoculation into the scaffold
d) A suitable volume of growth factors or pre-growth factors including but not limited to the Bone Morphogenic Proteins, Transforming Growth Factor Beta and Vascular Endothelial Growth Factor.
e) Written or Graphical Instructions on how to perform any or all of the relevant operations to restore bone stock in a body area with bone loss.

Preferably, the kit contains at least one growth factor, and even more preferably, bone biomimetic crystals that may be pre-placed within the scaffold within the kit.

Even more preferably, the at least one growth factor is an osteoblast growth factor.

In a further form, the osteoblast precursor cells are present in the bone growth paste of the invention.

In another form, stem cells or umbilical cord blood cells are present in the bone growth paste of the invention.

In yet a further broad form, the present invention relates to bone graft material, suitable for transplantation into a patient requiring said graft, said bone comprising:
a) a scaffold housing bone tissue; and
b) a blood supply comprising one or more blood vessels for suturing into a region in the patients body where the bone graft is required;

wherein said bone graft material has been manufactured according to the method of:
a) providing a scaffold for the bone graft material;
b) inoculating the scaffold with osteoblast precursor cells;
c) implanting the scaffold subcutaneously, subperiosteally or into fat or muscle tissue in a host;
d) harvesting the scaffold housing the bone tissue and a portion of a blood supply of the replacement bone when sufficient formation and angiogenesis of replacement bone has occurred.

The present invention is thus provided. Various features, subcombinations and combinations of the invention can be employed with or without reference to other features, subcombinations or combinations, and numerous adaptations and modifications can be effected within the spirit of the invention, and the literal claim scope of the invention is particularly pointed out and distinctly claimed as below.

In order that the invention may be readily understood and put into practical effect, particular preferred embodiments will now be described by way of the following non-limiting example.

### EXAMPLES

### Example 1

Replacement of Femoral Shaft

A patient is positioned in a supine position, however, an alternative position may be provided by positioning the patient on their side. A lateral approach is used. A pelvic holder may be employed.

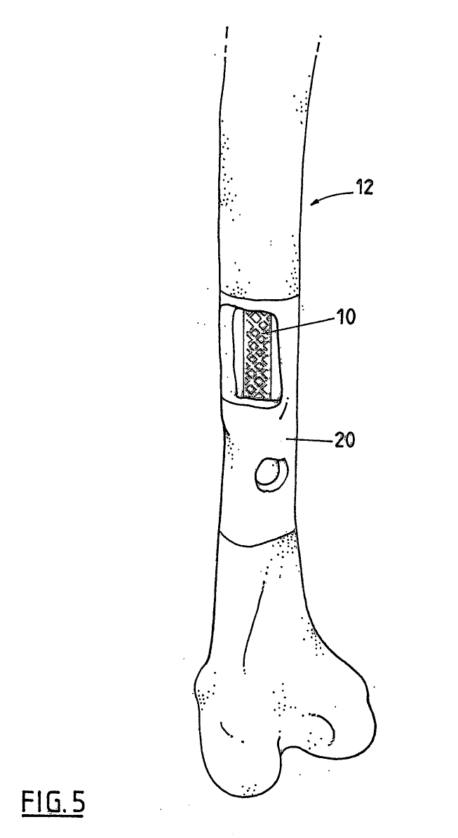

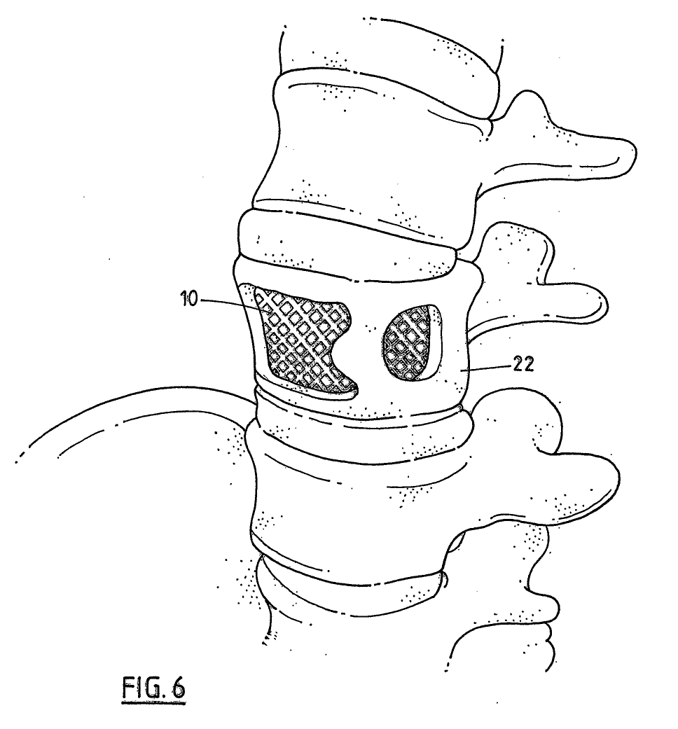

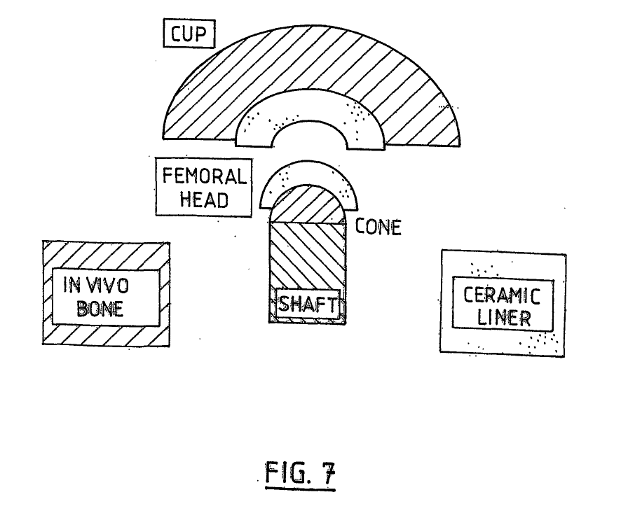

The operative technique firstly involves determination of the size of the defect to be filled/reconstructed from imaging studies (CAD CAM, X-ray, CT and MRI). An example of a scaffold 10 suitable for use in a replacement femoral 20 shaft is shown in FIG. 5, while FIG. 6 illustrates a suitable scaffold 10 for in vivo growth of a replacement vertebral disc 22.

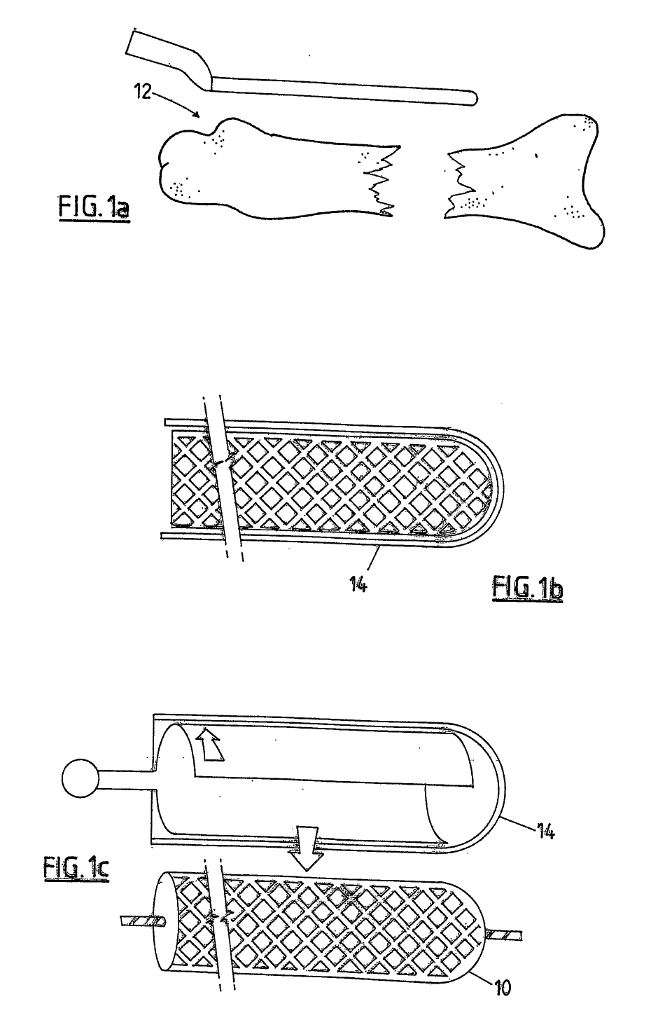

A standard minimally invasive surgical (MIS) incision is made (FIG. 1A), with dimensions of 5-cm over the lateral femur 12, directed posteriorly from the prominence of the greater trochanter. The fascia is then incised, and blunt dissected through to the lateral muscle mass.

The MIS Instrument 14 is then loaded (FIG. 1B) with the scaffold 10 (impregnated with bone paste and cells), and then it is introduced into the muscle mass (via advancing it in a similar mode to a tendon stripper). It is pushed distally along the lateral side of the femur 12, while an image intensifier is used to determine its positions. When it is alongside the bony defect (which is to be later filled), the scaffold 10 implant is released from the MIS Instrument 14 (FIG. 1C).

The scaffold 10 is left for six to eight weeks, and bony growth within the scaffold 10 is reviewed with CT imaging, bone scintigraphy or biopsy.

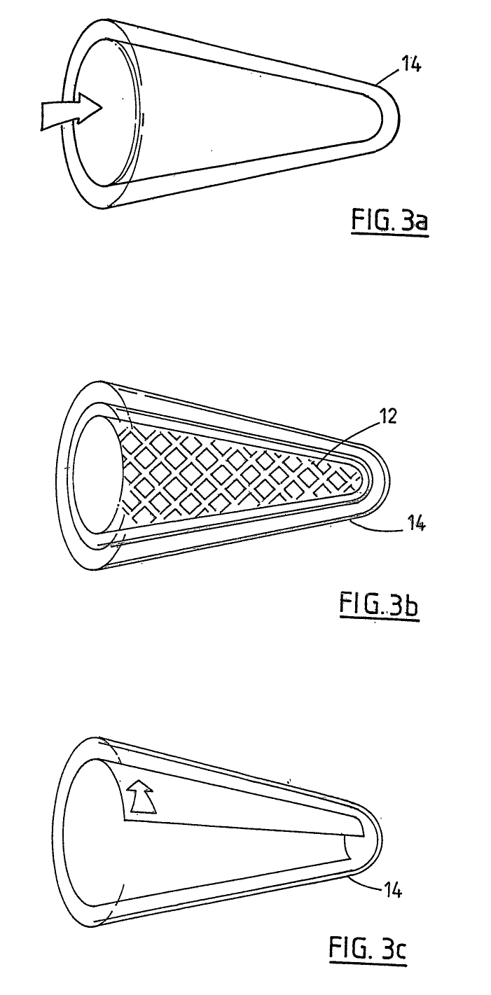

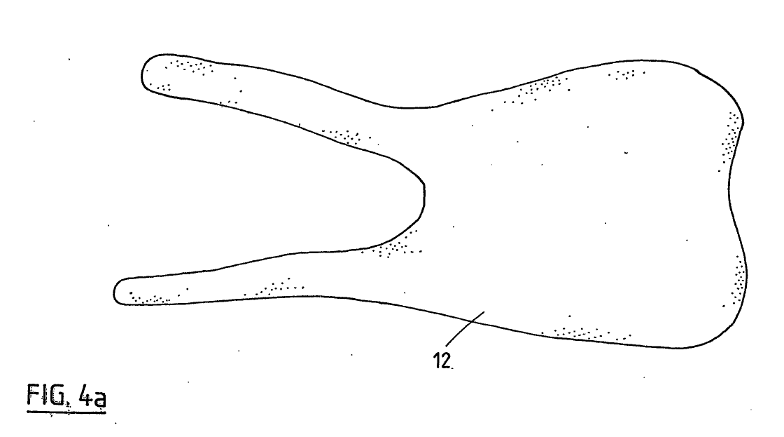

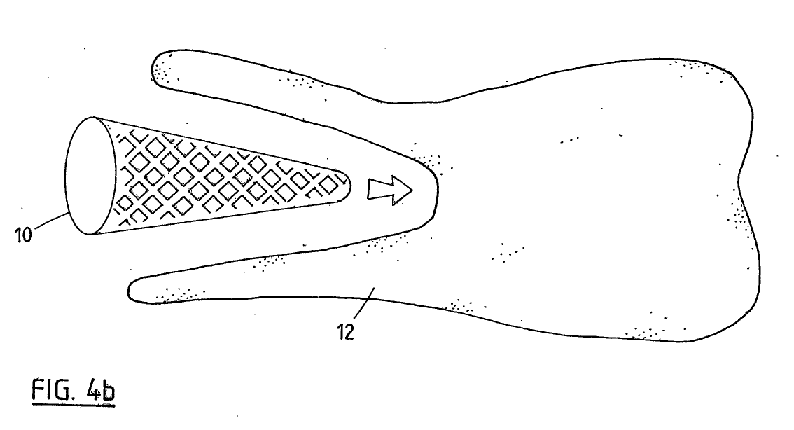

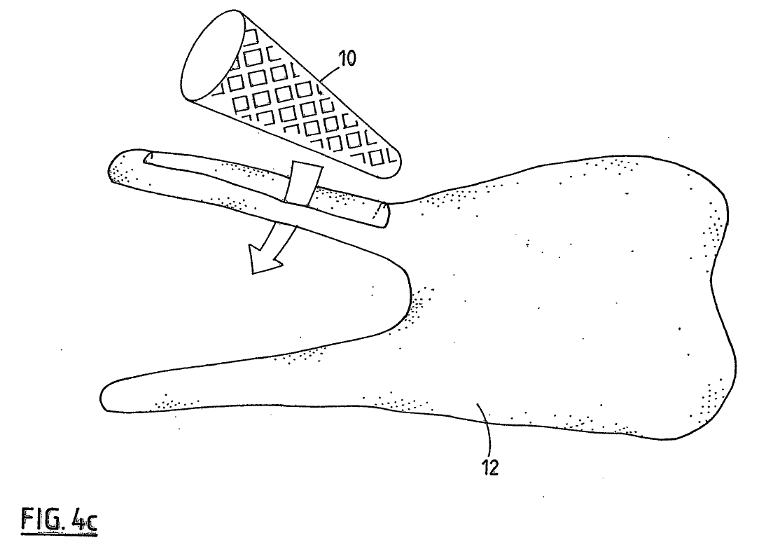

In clinical situations involving loss of bone stock, loss following failed joint replacement surgery, cone-shaped scaffolds 10 can be used to fill the bone stock loss of the upper or lower femur (FIGS. 3A to 3C, showing loading and unloading of cone-shaped scaffold 10 from MIS instrument 14 and FIGS. 4A to 4C, showing insertion of cone-shaped scaffold 10 into a defect in the upper femur 12 in the clinical situation of bone stock loss after failed joint replacement surgery).

After 6 to 8 weeks, the patient returns to the surgeon, and use the same or an extended lateral incision is made in the thigh using MIS instruments 14.

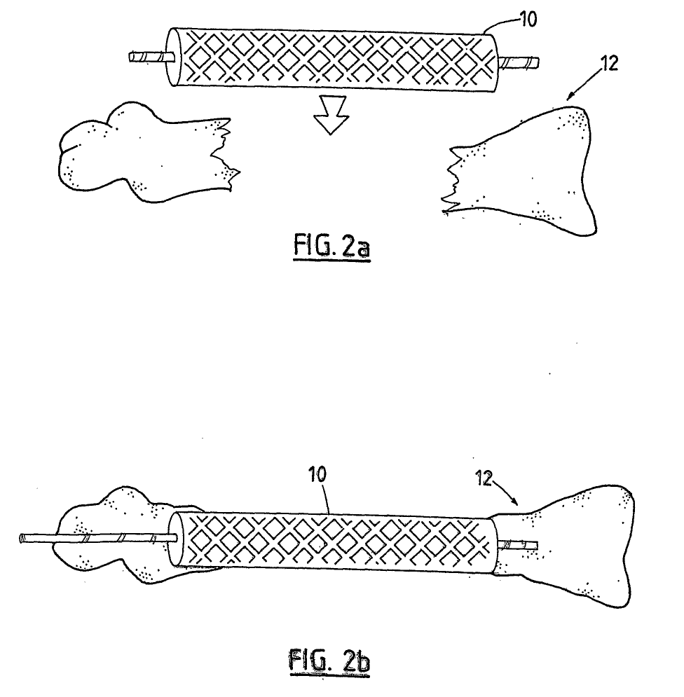

The scaffold 10 and bony replacement material is mobilised (FIG. 2A), without stripping the muscle mass (ie NOT disturbing the vascular supply), and moved into location the desired location (ie. the site of the bone defect) (FIG. 2B). An image intensifier is useful in determining the precise final location of the scaffold 10 and replacement bone tissue.

The implant is anchored at either end with a plate or rods or any other standard method known in the art.

Confirmation of the stability of the implant and post-operative protection may be required for several weeks following the procedure.

Bony healing is generally monitored via Radiographs, Bone scan (or Biopsy).

Postoperative rehabilitation involves application of a cryocuff/ICEMAN to the surgical region in the recovery room, and for the next 48 hours.

Persons skilled in the art will appreciate that numerous variations and modifications will become apparent. All such variations and modifications which become apparent to persons skilled in the art, should be considered to fall within the spirit and scope that the invention broadly hereinbefore described.

1. A method for growing replacement bone tissue for a patient, said methods characterised by comprising:
a. providing a scaffold for the replacement bone tissue;
b. inoculating the scaffold with osteoblast precursor cells;
c. implanting the scaffold in a location close to a site where replacement bone is required, wherein the location comprises a subcutaneous or subperiosteal compartment, a muscle or fat tissue;
d. allowing osteogenesis and angiogenesis of the replacement bone tissue;
e. relocating the replacement bone tissue and its substantially intact blood supply to the site where replacement bone is required in the patient.

2. Method according to claim 1, characterised in that inoculating the scaffold further involves inserting hydroxyapafite crystals into the scaffold.

3. Method according to claim 2, characterised in that the hydroxyapatite crystals are provided as bone mineral blocks or specially shaped crystals.

4. Method according to claim 1 characterized in that the osteoblast precursor cells are a mixture of bone marrow cells.

5. Method according to claim 1 characterized in that the osteoblast precursor cells are derived from a mixture of bone marrow cells.

6. Method according to claim 1 characterized in that the osteoblast precursor cells are mesenchymal stem cells.

7. Method according to claim 1 characterized in that the osteoblast precursor cells are hematopoietic stem cells.

8. Method according to claim 7, characterised in that the haematopoietic stem cells are derived from monocyte precursor cells.

9. Method according to claim 1 characterized in that the osteoblast precursor cells are adult stem cells or embryonic stem cells isolated from an embryo of the host species.

10. Method according to claim 1 characterized in that the osteoblast precursor cells are totipotent stem cells isolated from a fertilized egg of the host species.

11. Method according to claim 1 characterized in that the osteoblast precursor cells are autologous with respect to the patient's tissue.

12. Method according to claim 1 characterized in that the osteoblast precursor cells are allogenic with respect to the patient's tissue.

13. Method according to claim 1 characterized in that inoculating the scaffold further includes providing at least one growth factor within the scaffold.

14. Method according to claim 13, characterised in that inoculating the scaffold further includes providing at least one growth factor on the outer surface of the scaffold.

15. Method according to claim 14, characterised in that at least one growth factor is selected from the group consisting of the bone morphogenetic protein (BMP) family members.

16. Method according to claim 15, characterised in that the BMP is BMP-2.

17. Method according to claim 15, characterised in that the BMP is BMP-7.

18. Method according to claim 15, characterised in that the osteoblast precursor cells are and growth factors are provided as a bone mineral paste, the bone mineral paste further comprising bone crystals.

19. Method according to claim 18, characterised in that the bone crystals are pre-shaped before being added to the bone paste.

20. Method according to claim 19, characterised in that the bone crystals are pre-shaped to be hexagonal.

21. Method according to claim 1 characterized in that the scaffold is a cone shape or a cup shape.

22. Method according to claim 21, characterised in that the cone or cup-shaped scaffold is used to replace lost or damaged bone resulting from failed hip or knee joint reconstruction or replacement.

23. Method according to claim 1 characterized in that anatomical modelling studies are performed for shaping the scaffold to optimize the scaffold shape to fit the site where replacement bone is required in the patient.

24. Method according to claim 23, characterised in that the anatomical modelling studies include computed tomography and/or selective laser melting technology.

25. Method according to claim 24, characterised in that the anatomical modelling studies further include use of computer-aided design.

26. Method according to claim 23, characterised in that the anatomical modelling studies include three-dimensional computed tomography and/or magnetic resonance imaging.

27. Method according to claim 1 characterized in that the scaffold is a suitable biocompatible and/or bioabsorbable material.

28. Method according to claim 27, characterised in that the biocompatible and/or bioabsorbable material is selected from titanium, stainless steel, zirconium oxide, ceramic tricalcium phosphate and polymers; or bioplastics and biopolymers—either existing or innovative materials; and whether transplanted, implanted, or injected; generated in situ or externally; or nanogenerated or nanoconstructed structures, or polymeric lattices.

29. Method according to claim 1, characterized in that the scaffold is titanium.

30. Method according to claim 1, characterized in that the scaffold has a mesh-like or matchstick shape and or structure.

31. Method according to claim 1 characterized in that the scaffold has a gel-like structure.

32. Method according to claim 30, characterised in that the scaffold has an inner mesh-like surface and a substantially complete outer surface, wherein the osteoblast precursor cells are injected into the interior of the inner, mesh-like surface of the scaffold through the substantially complete outer surface.

33. (canceled)

34. Method for growing bone for a bone graft in a patient, characterized in that the method involves the steps of:
a) providing a scaffold for the replacement bone tissue;
b) inoculating the scaffold with osteoblast precursor cells; and
c) implanting the scaffold to a site where replacement bone tissue is required in the patient.

35. Method according to claim 34, characterised in that the scaffold is implanted into a region where failed joint replacement surgery has resulted in bone stock loss.

36. Method according to claim 35, characterised in that the failed joint replacement surgery involved replacement and/or reconstruction of the hip or knee joints.

37. Method according to claim 36, characterised in that the scaffold is a cone shape or a cup shape.

38. Method according to claim 37, characterised in that the cone or cup-shaped scaffold is used to replace lost or damaged bone resulting from hip or knee joint reconstruction or replacement.

39. A kit for growing replacement bone for a patient, the kit characterised by comprising:
a) a scaffold suitable for supporting bone growth subcutaneously, subperiosteally or within fat or muscle tissue of a host; and
b) a source osteoblast precursor cells.

40. The kit according to claim 39, characterised in that the scaffold is a biocompatible and/or bioabsorbable material.

41. The kit according to claim 39, characterised in that the scaffold is titanium.

42. The kit according to claim 39, characterised in that the scaffold comprises an inner, mesh-like structure for housing the osteoblastic precursor cells and a substantially complete outer surface through which the osteoblastic precursor cells are injected into the mesh-like structure that houses said cells.

43. The kit according to claim 39, further including at least one osteoblast growth factor.

44. The kit according to claim 39, further including hydroxyapatite crystals suitable for placement in the scaffold.

45. The kit according to claim 39, further including hydroxyapatite crystals pre-placed within the scaffold.

46. The kit according to claim 45, characterised in that the hydroxyapatite crystals are present as bone mineral blocks.

47. The kit according to claim 45, characterised in that the osteoblastic precursor cells, the at least one osteoblast growth factor and the hydroxyapatite bone crystals are provided as a bone paste or gel.

48. The kit according to 45 characterised in that the hydroxyapatite bone crystals are pre-shaped.

49. The kit according to claim 48, characterised in that the hydroxyapatite bone crystals are hexagonal.

50-82. (canceled)
